(19) United States
(12) Patent Application Publication
(10) Pub. No.: US 2024/0311860 A1
(43) Pub. Date: Sep. 19, 2024

(54) NOVEL SYSTEMS AND METHODS FOR PROVIDING MEDICAL TREATMENTS WITH RECONCILED PAYMENTS

(71) Applicant: Joseph Alan Epstein, Pleasanton, CA (US)

(72) Inventor: Joseph Alan Epstein, Pleasanton, CA (US)

(21) Appl. No.: 18/671,816

(22) Filed: May 22, 2024

Related U.S. Application Data
(63) Continuation-in-part of application No. 16/508,258, filed on Jul. 10, 2019.
(60) Provisional application No. 62/696,059, filed on Jul. 10, 2018.

(51) Int. Cl. G06Q 30/0201; G06Q 30/0204; G16H 80/00

(52) U.S. Cl. CPC G06Q 30/0206; G06Q 30/0205; G16H 80/00

(57) ABSTRACT

A method and system of determining a cost of medical treatment and providing said treatment to patients according to one embodiment, based upon the medical needs of the member patients and the amount of funds the members have had contributed on their behalf into the membership plan, including insurance methods. Patients are provided medical treatment directly by primary and basic specialty care physicians according in part to their membership levels. The treatments available are adjustable retrospectively and can be set prospectively by service assignment relating the patient's needs and the pool of providers' abilities, licensure, and equipment. In some embodiments, the reimbursements are backed by a further insurance product operated as a stop-loss or reinsurance specifically tied into the nature of the division of treatment among providers as provided for by the invention.

### CROSS REFERENCE TO RELATED APPLICATION

This application is a continuation-in-part (CIP) of U.S. patent application Ser. No. 16/508,258 filed on Jul. 10, 2019, and entitled “Novel Systems and Methods for Providing Medical Treatments With Reconciled Payments,” which further claims the benefit of provisional patent application Ser. No. 62/696,059, filed Jul. 10, 2018 by the same inventor. All mentioned U.S. applications are hereby incorporated by reference.

### BACKGROUND OF THE INVENTION

### Field of the Invention

The present invention relates to the field of delivering medical care and treatments to one or more patients, and in the collection and reconciliation of payments and service eligibility for said patient, including insurance methods.

### Description of the Prior Art

The existing regime for determining whether a patient is eligible for a particular treatment, providing the treatment, and collecting payment in this country is carried out in such a way as to both increase complexity in all stages and to reduce the quality and availability of care. The soaring costs of medical treatment, and thus medical insurance to underwrite the treatment, is leading to aggressive attempts to solve our problems by innovating in the way patients enroll for care, receive care, and have their care paid for-innovations such as capitation, direct primary care, private primary care, accountable care organizations. But these innovations have not really taken hold, mostly because they have not solved the problem-a problem that is fundamental to the way treatment is tied to the collection of fees.

Our modern practice of medicine has become unruly, inefficient, and overly expensive. One main reason for this is simple to observe but incredibly difficult to cure: paying providers for each procedure they do leads to conflicts of incentives that drive providers to want to provide care inefficiently (measured by cost, or outcomes, or both) and drive insurers to want to ration care.

How did this problem arise? Because the costs that insurance companies assign to procedures, via reimbursement codes, has very little relationship to the actual cost of the procedures. Fixed overhead gets falsely mixed into per-procedure charges, as providers look to ensure they cover their costs if there aren't enough procedures. And then, if there are enough to cover the costs, they suddenly have a perverse incentive to push these procedures even harder, as each procedure, having already covered the fixed costs, can lead to the percentage established by the provider for fixed charges becoming pure profit (or excess revenue for nonprofits, a particularly dangerous thing because it threatens their state nonprofit status). The family of a baby in San Francisco who visited the emergency department at the Zuckerberg Chan SF General Hospital was billed over $18,000 for a three-hour treatment regimen that consisted of being given a warm bottle and a nap. The excuse: to cover the high fixed costs of having a trauma center, they charge almost $16,000 per visit, no matter what the reason and no matter what services were rendered. The patient is then forced to face a perverse disincentive to visit the doctor, knowing that they'll be slammed with fees not reasonable in the face of the services provided: anything from a $100 bandage to multiple thousands of dollars for just walking through the door. The doctor, who is ethically and legally required to offer procedures to each patient without regard to cost, becomes an inadvertent money-printing machine. And the hospital, who often is a nonprofit who does not want the penalties for excess profit that they are now getting hit with regularly, is forced to look like the bad guy, the greedy storeowner who charges customers just for the privilege of being open, when what they really want to do is make sure they don't go bankrupt in a downturn.

The irony of it is that this problem is created by the insurance company, the one entity the public tends to view reasonably, and worse, the one entity who has to cover those fees and has every incentive to make them go away. Or rather, the problem is created by the method the insurance company uses to fund medical care: reimbursements tied to procedure codes.

So, why do we have procedure codes and tied reimbursements? Because medical providers do not want to provide services independent of actual costs, and because insurance companies do not want to pay for medically unnecessary procedures. Providers need, in this system, to bill for everything they do. Skip a few and they may be in danger of not covering their costs. SF General really does have to pay all of their ED physicians, whether they are used or not. So providers feel they have no room to not bill every variable cost as aggressively as possible, in hopes that they never fail to cover their fixed costs. On the other side, insurance companies are tantalizing targets. Unethical medical providers can easily construct ways of doubling their profits by overprescribing mostly harmless but mostly unnecessary procedures, knowing they can bill each one out. This causes insurance companies to double down on the procedure codes, demanding increasing specificity and control so that their own physician reviewers can re-deduce the entire medical examination and determine for themselves if they are being taken for a ride.

FIG. 1 (prior art) diagrams the interplay of conventional systems. Procedure codes are costed out in 110, taking into account the fixed costs 105 necessary to fund a clinic capable of delivering the procedure, and all other fixed costs, and the variable costs 100 related to the specific procedure. These costs are output as the cost 135 for each code, each cost containing its variable costs 136 and fixed costs 137 as per the costing analysis 110. Once the doctors or other practitioners 120 through the service provider 130 provide the services to the patients 135, a billing system 145 (usually a part of the electronic health records) finds the procedure codes the doctors or other practitioners entered to record what they did, and generates the copay 126 and coinsurance 127 that the patient owes as an invoice to the patient, sending the rest to the insurer or final payor for reimbursement 150. This tight cycle of billing-reimbursement occurs throughout the plan year. Once the plan is coming up for renewal, the negotiation 155 occurs with the provider to adjust the procedure costing and the resulting costs to a level the insurance company or payor is willing to pay, thus requiring the provider to further bury costs and create distorted output rate tables for reimbursements. Generally, although at a high level guided by human interaction, the details of the thousands of rate codes and the estimation of whether the next proposed offer in negotiations will produce sufficient revenue for the provider and sufficient profit for the insurer is determined by complex computer models far exceeding the capabilities of any one or group of humans.

This self-reinforcing cycle only entrenches the system further, giving not just the illusion of stability but falsely encouraging participants that the solution is to fine-tune and redouble efforts within the system—as they are attracted to the complexity and feel that that itself must be the source of the inefficiency—rather than restructuring parts of the system itself.

FIG. 2 (prior art) shows how the output of the rate setting processes leads to excess profits. The same rate setting processes (200-215) produce the rates 220. When the actual list of billed procedures for an accounting period are laid out as in 225, and assigned to fixed costs 250 and variable costs 230, the ultimate overcharging to avoid any risk leads to an excess “profit” of 240 for the over-hedged fixed costs. This is a major problem: it has caused nonprofit organizations to become over-flush with cash and having their nonprofit status stripped or threatened.

Some innovation has occurred in the area of fixed or membership-based services. These services work by providing primary care for a fixed cost, thus solving the problem of how to keep the lights on or how a patient can now where to go. Fixed services operate using accountable care organizations or capitation agreements, where the insurance company basically gives the provider fixed cash per patient per year to force the provider to assume some or all of the additional risk, should a patient need more care than the provider has been paid in advance. Ideally, the provider will overcharge for each patient just enough to absorb that risk, while still satisfying the payor's demands. But let's be clear here: the payor has the power and sets the price, and the provider hopes to be stingy enough by rationing care (while not triggering the ACO quality metrics designed to detect for this and punish the provider) or lucky enough in their guess of the prices to absorb any actual blow. Private versions of this work as a membership-based scheme: the patient is the payor, and buys a membership to a primary care clinic. That limits the risk of the provider: primary care has a natural cap in costs, since it's a tiny fraction of actual care. In fact, it is nearly a luxury item—many patients see a primary care physician once a year for the mandatory annual and the flu shot, and those who have greater needs usually fall on the other end of the barbell and receive most of their treatments from specialists—and it is no surprise that these clinics provide luxury furnishings and the latest gizmos to try to convey a sense to the patient of how much luxury they've paid for. In other words, primary care alone is easy to address: primary care physicians serve the role that service advisors do at a car repair shop. GPs need a basic understanding of everything medical, but in the prior art are judged as much on bedside manner and are expected to have others do the hard work. However, unlike car repair, where service advisors are desk workers and the repairmen are blue-collar workers in the garage, in medicine, a doctor is a doctor is a doctor. They all operate at roughly the same level of competence. There's no reason that a primary care physician should be a gateway to real care, rather than the source of real care herself.

What the prior art fails to do is to take specialty care into account at the outset. They treat it like it is special, thus the name, even though it dominates the health care spend for large population groups. Therefore, bringing specialty care into the fold up front is key to managing total procedure cost. The primary care physician needs to regain their role as the coordinator of the total chronic and routine care of the patient. The PCP needs to be the patient expert—and needs to be incentivized and have enough time made available to perform the background and direct investigational work needed to become that expert and maintain that expertise. Just as the police appoint a lead detective to solve a crime or a series of crimes, the health care system should have someone to perform the role of lead investigator for the patient's health. It should not be specialized to the point of balkanization, as it is forced to be by the perverse incentives of the standard insurance reimbursement scheme.

The degree of specialty care services rendered and their costs today exceed primary care for all patients who have any real need of physician care. If specialty care occurs outside of the system of primary care, then the bulk of the procedure costs cannot be addressed, leaving the prior art as half-baked at best.

The present invention addresses these problems by disclosing a novel way of providing primary and specialty care to patients, determining whether they should be able to receive care and from whom, and ensuring the proper reconciliation of the process, including novel insurance methods.

### SUMMARY

In accordance with one embodiment, a method and system for treating patients, determining the level and specific items of care the patient is eligible for based on the plan he or she is enrolled in, ensuring an adequate availability of baseline services, and matching up with providers, services, and treatment options.

In one embodiment, a method is provided for making specialty medical treatment available to patients. The method comprises operating a first machine learning system to certify primary and basic specialty care providers, assigning to the patient a primary and basic specialty care provider that provides primary care and basic specialty care procedures and services, and operating a computer to calculate fees for the primary care and basic specialty care procedures and services based on patient populations. The method also comprises providing to the patient, by the assigned primary and basic specialty care provider, any of the primary care and basic specialty care procedures and services, operating a second machine learning system that automatically triggers a consultation with a consulting specialist provider based on the primary care and basic specialty care procedures and services provided to the patient, and operating a telepresence system to conduct the consultation that provides communication between the patient, the primary and basic specialty care provider, and the consulting specialist provider to provide specialty medical treatment to the patient when the specialty medical treatment is not part of the primary care and basic specialty care procedures and services.

In one embodiment, a system is provided for making specialty medical treatment available to patients. The system comprises a first machine learning system that certifies primary and basic specialty care providers, a primary and basic specialty care assigner, which assigns to a patient a primary and basic specialty care provider, and a services assigner, which assigns to the assigned primary and basic specialty care provider a set of procedures and services that comprise both primary care procedures and specialty care procedures that the provider can perform on the patient under a medical plan. The system also comprises a computer to calculate fees for the set of procedures and services based on patient populations, a second machine learning system that automatically triggers a consultation with a consulting specialist provider, and a telepresence system to conduct the consultation that provides communication between the patient, the primary and basic specialty care provider, and a consulting specialist provider to provide specialty medical treatment to the patient when the specialty medical treatment is not part of the primary care and basic specialty care procedures and services.

Additional features and benefits of the exemplary embodiments of the present invention will become apparent from the detailed description, figures and claims set forth below.

### BRIEF DESCRIPTION OF THE DRAWINGS

FIG. 1 is a diagram of the prior art, illustrating the cost breakdown for CPT (current procedural terminology) procedures and services.

FIG. 2 is a diagram of the prior art, further illustrating the cost breakdown for CPT procedures and services.

FIG. 3 is a diagram of some embodiments of the invention, illustrating the breakdown of services between providers and the connections between them to ensure coordinated care.

FIG. 4 is a diagram of some embodiments of the invention, illustrating the validation of the membership of the patient at the points of service, and the establishment of reimbursement flows that are sufficient for the patient load.

FIG. 5 is a diagram of some embodiments of the invention, illustrating the service assignment system.

FIG. 6 is a diagram of the output of an embodiment of the invention, assigning to a PBSCP practice a core set of basic specialty care procedures to be performed.

FIG. 7 shows a diagram of a certification system that utilizes machine learning to certify primary and basic specialty care physicians.

FIG. 8 shows a diagram of a neural network for use in the certification system shown in FIG. 7.

FIG. 9 shows an embodiment of a front-line processing system that utilizes machine learning for use in the embodiment of the system shown in FIG. 3.

FIG. 10 shows a diagram of a neural network for use in the front-line processing system shown in FIG. 9.

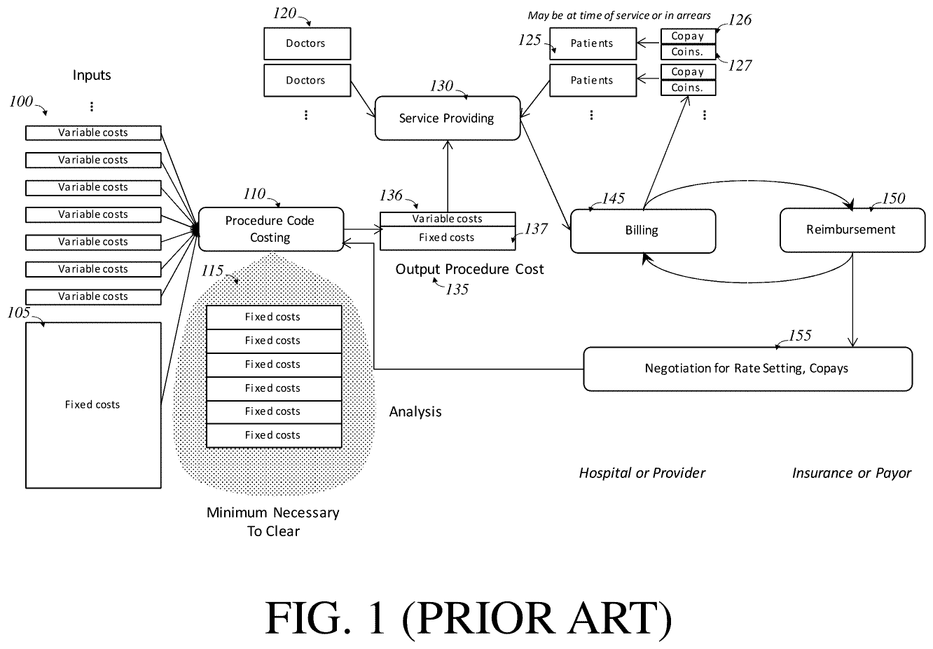

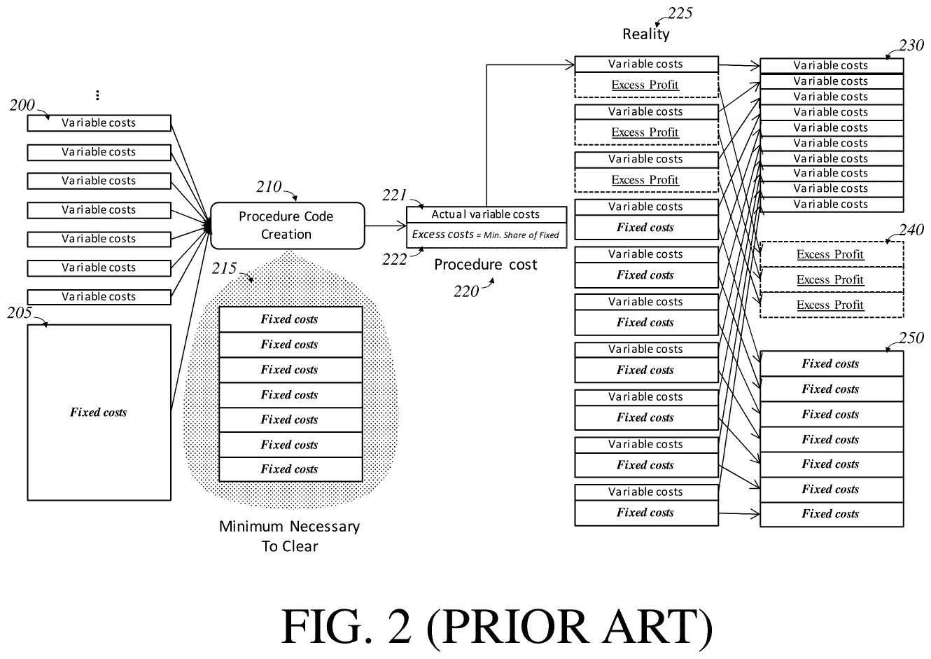

### DETAILED DESCRIPTION

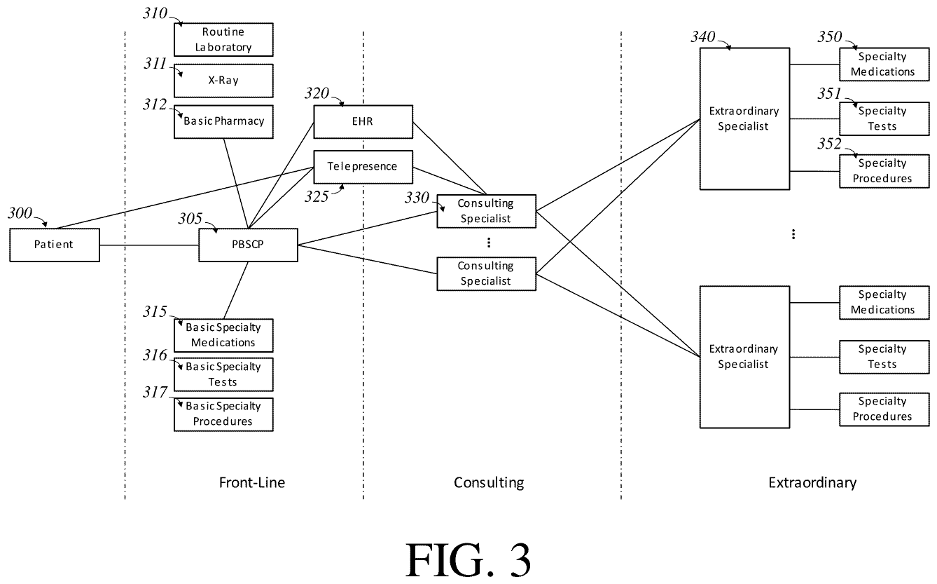

FIG. 3 diagrams some embodiments by providing consulting specialists. The patient 300 seeks treatment from a Primary and Basic Specialty Care Provider 305 (PBSCP), to which she is assigned (such as by her choice, her needs, availability, or any other mechanism). (Note that a provider may be a solo practitioner, or multiple practitioners operating together to cover the needed services, depending on the particular abilities of the practitioners.) The PBSCP differs from a traditional PCP by providing not only primary care but basic specialty care. In some embodiments, this includes at least one of routine laboratory work 310, X-Rays 311, basic pharmacy 312, basic specialty medications 315 (such as appropriate for endocrinology, allergy, cardiology, or pulmonology). The goal is to have the PBSCP practitioner operate closer to the top of his or her license, thus being able to handle significantly more than a traditional PCP, who must refer out many of the procedures. In some embodiments, PBSCP are chosen from a pool of providers trained in extra critical thinking skills. In some embodiments, they are chosen from previous specialty care providers. Some embodiments allow them to operate by themselves without consulting providers for their specialty. Some embodiments retain the practitioners as CSPs (defined below) for other PBSCPs as well.

The PBSCP (or for other embodiments with less care provided by the primary practitioner, the GP or PCP) may call for a consult from a consulting specialist provider (CSP) 330. A CSP practitioner is a full specialist—a specialist capable of providing more than basic specialty care—who sits in on the case when needed. In some embodiments, the CSPs are located in a cost-efficient manner, such as having them in a central location per locality, per state, per region, or nationwide. In some embodiments, this occurs when the PBSCP practitioner 305 determines that she needs a consult to explore a condition that she does not feel comfortable taking alone, or because the patient requested it, or when it is clear that the patient would be comforted by having the consulting specialist present, the physician calls for the consult. In some embodiments, the consult is triggered automatically through an automation or analysis system: some request the consult immediately; some schedule it; some place it into a queue for later scheduling. In some embodiments, the consultation occurs via telepresence 325 for the CSP practitioner 330, who has access to the EHR 320 and a patient facing display, if available. The specialist will then be able to provide the further handling and advice and offer the GP or (P)BSCP directions, order further tests which can be performed immediately or in the presence of the patient or refer to an extraordinary specialist provider (ESP). One embodiment continues the notion that the PBSCP (or PCP and BSCP in concert) is the patient expert, the one main point of contact, and the person responsible for understanding in a complete medical way what the patient's situation in and how it will proceed. In this case, the (P)BSCP may call together a patient consulting board of specialists who may remain throughout the case, to be managed and altered by the PBSCP.

For this additional consulting contact, one embodiment is for the consulting specialty services to be funded from an inclusive charge, in the same way as the (P)BSCP is funded. In that method, there is no per contact fee, and the consultations occur as needed medically. In another embodiment, the access to some or all of the CSPs is based on a separate subscription or inclusive fee. In another embodiment, the CSPs are billed out on a procedure code to an insurance or self-insured payor. The advantages of having an inclusive fee and service is to give the patient one continuous medical experience, with the CSPs able to also explore the patient's condition in the same manner as the (P)BSCP. Even though the CSPs may be loaded based on different actuarial tables, it may be desirable to build in a similar lower loading rate to ensure that the CSPs have the ability to perform medical care first.

If the patient needs extraordinary specialty care (beyond the reach of CSPs if present), the patient may then be referred out to that care. In one embodiment, the CSPs have continued EHR integration with the extraordinary care providers 340. In another, the (P)BSCPs and/or CSPs have practicing rights at those facilities and can lead or provide the services. A possible advantage of that is that the (P)BSCPs (and CSPs) time may already be included in the billing function of the method performed in some embodiments, and the additional accrued costs can be limited to facilities and support staff to the largest extent possible. Because of the application of specialty care closer to the patient, the number of referrals may be significantly less and, outside of a few routine automatic referrals (OB/GYN, for example), may tend to line up with emergency and catastrophic care, and thus may have more favorable usage statistics.

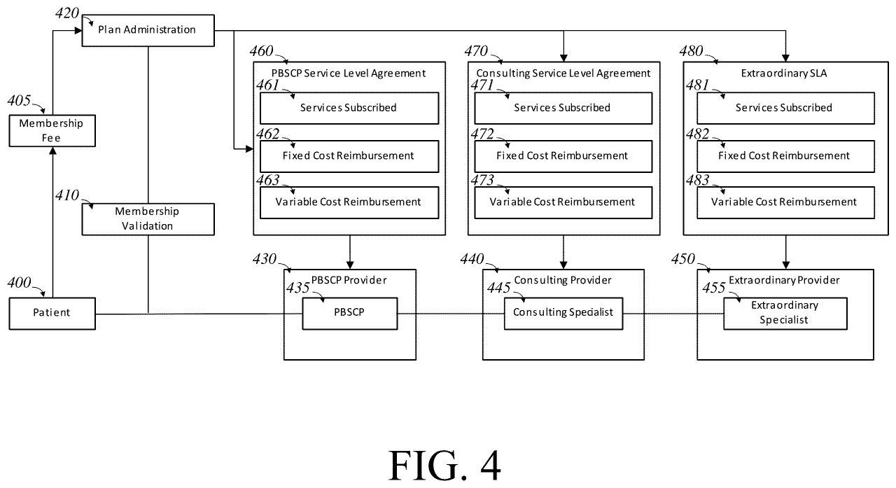

FIG. 4 diagrams some embodiments that operate with consolidated enrollment and billing. The plan administrator 420 collects from the payor (the patient 400, the employer or plan sponsor, an insurance carrier, or their agents or administrators, alone or in combination using a sliding scale, fixed percentages, waterfall, or other) a membership fee 405. This fee entitles the patient (and/or family) access to services. One embodiment is to provide unlimited access; another is to have some limits in place. Other embodiments provide limits by number or type of access or correlated with the conditions or based on need. There's no experimentation here in choosing limits: it is merely a standard operation based on the cost/actuarial balance. The fees collected in some embodiments are related to the fixed costs needed to operate the PBSCP: one embodiment collects at least enough funds to offset the patient's share of the fixed costs of the PBSCP; another uses a multiple (greater or less than 1) of the patient's share of the fixed costs, thus allowing discounts or surcharges. This multiple can vary between patients.

The plan administrator ensure that care is provided to for the patient. One embodiment is for the patient (or his/her family) to be assigned to one PBSCP 435, who may either be a solo practitioner in her own provider 430 or be a part of a larger provider organization. This physician is paid (in part or whole—subsidies are allowed) by the plan administrator from the funds collected from the payor(s). One embodiment controls the physician's load—the number of patients the physician sees—to ensure that the physician has enough patients to pay for his salary and share of tools and office space, but not so much as to starve him of the time he needs to perform significant open-ended diagnostic inquiry into the patient. Another embodiment is to provide a fixed number: one is to use X (say 500) patients per physician; another is to use X immediate families per physician. The PBSCP is provided the medical tools and training necessary to perform both the primary and basic specialty care.

When the patient accesses care, his membership is validated by 410, which confirms the membership with the plan administration. The provider of care is also provided, for each patient, the service level agreement (SLA) 460, 470, or 480. In some embodiments, a joint SLA exists; in others (such as shown in the figure), each provider type receives the SLA appropriate for the type. In some embodiments, the SLA is presented as a uniform entity provided all at once; in some embodiments, the component pieces are provided separately or at different times. In some embodiments, the SLA is provided to each provider upon assignment of the patient: in some embodiments, the PBSCP is provided the SLA upon patient enrollment or assignment to the primary provider; in some, the first contact with the patient establishes the relationship and thus provides the SLA or a part of it. In some embodiments, the SLA contains the services subscribed to by the patient, thus allowing the provider to determine if the patient is eligible to receive particular or general services during the treatment process. In some embodiments, the SLA contains payment or a reference to a promise to pay for the fixed costs 462 etc. and variable costs 463 etc. In some embodiments, the fixed costs are not provided per patient but per the provider agreeing to take patients: these fixed cost reimbursements may be set based on a number of factors, including at least one of the number of patients, the class of patients, the degree of care the class or patient needs, the location of the patients, the patients' own fee rates or contributions to the plan. An advantage of these methods is that the reimbursements can be provided up front in large part—or entirely if the model employed by the plan is to sufficiently pay up front for the expected variable costs, which are now likely to be smaller. This can allow for providers to have their fixed and variable costs sufficiently covered, thereby reducing or eliminating the risk of being a provider for such a plan.

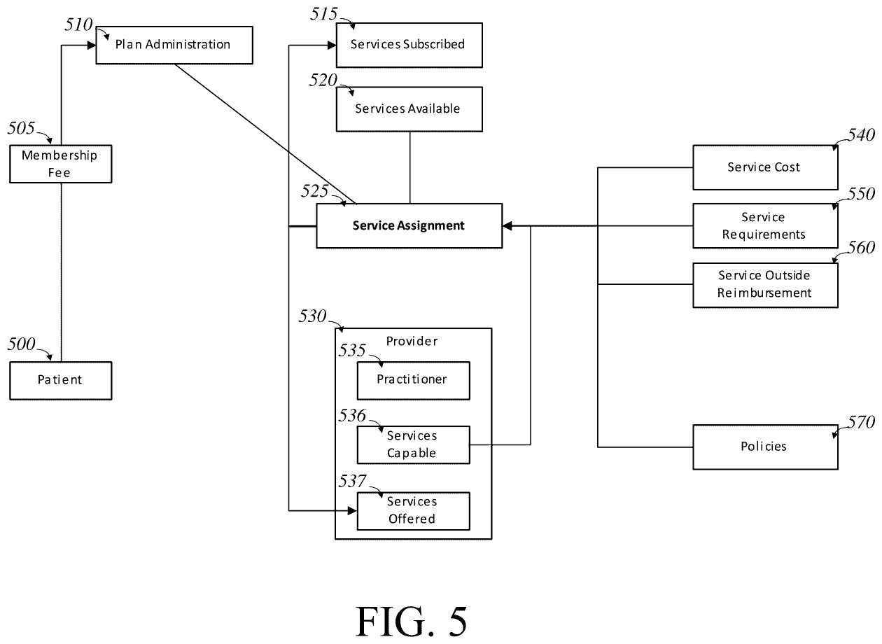

FIG. 5 illustrates the determination of the care to be provided to a patient by the PBSCP for some embodiments. The plan administrator 510 performs or has performed on its behalf the function of service assignment 525. Service assignment takes into account, for each provider 530, the practitioners 535 they have and the services they are capable of 536. In some embodiments, the services they are capable of are constrained by the training, licensure, and education level of the practitioners and the equipment they have on hand. In some embodiments the service assignment also takes into account the service cost 540 (fixed and variable) and the requirements necessary to perform the service 550. In some embodiments, it also takes into account the service's outside reimbursement rate 560, such as the rate it would cost for the plan to pay for the service on a fee-for-service basis instead in the open market or through a network. In some embodiments, the services are also influenced by policies 570: some embodiments use stated policies of minimum or acceptable standards of care; some embodiments use policies tied to the service levels that patients have subscribed to. These policies may require standard levels of service even with no patient input, and thus can set a baseline (or multiple baselines in the case of tiered offerings). In some embodiments, the services in need are filtered by the capabilities and requirements, to produce a list of services available 520. In some embodiments, the assignment 525 populates the services that are offered by the provider 537. In some embodiments, this is a strict subset of the services capable 536 used in the assignment. In some embodiments, the assignment can instruct the provider to acquire greater capabilities in the outputting of the services offered 537. The service assignment also takes the patient's membership via administration and produce, for that patient, the services that the patient is subscribed to. In some embodiments, the services are referred to by their CPT codes. In some embodiments, the outside rate 560 is pegged to the Medicare reimbursement rate for the procedures. In some embodiments, the risk of the practitioner providing the service is taken into account. It is clear that such assignment will require sufficient computational resources, but steps can be taken to reduce the impact by, for example, establishing a baseline of service that will require the PBSCP to offer those treatments without regard for their capabilities (and thus shifting the burden of ensuring that they are capable to the sign up process for the PBSCP rather than the services assignment itself), dividing the assignment up between patents around region or class of patient, using computer resources dedicated to those regions or classes, and limiting the recalculation to changes in the service level requested by the plan and avoiding mid-plan-year changes.

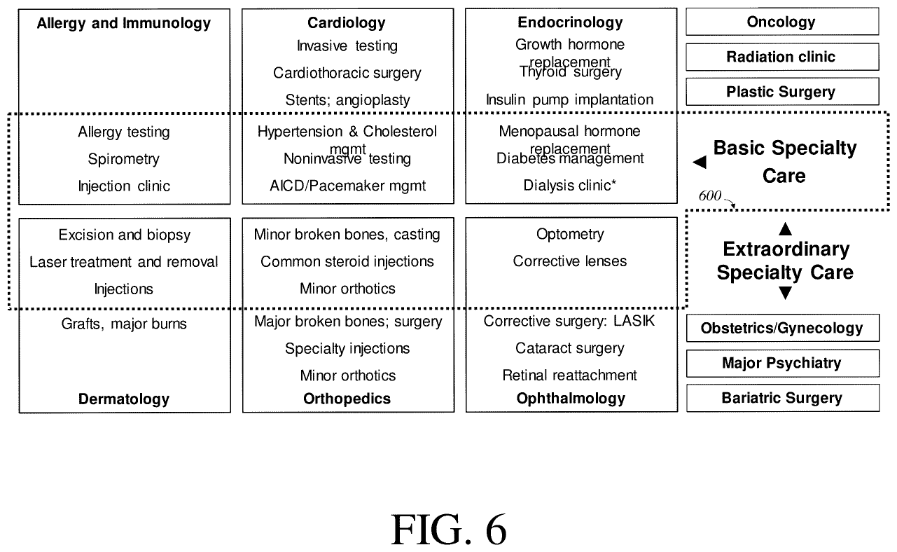

FIG. 6 shows an output of an embodiment of the invention showing a typical assignment of services to the PBSCP based on an expected competency in line with licensure in California, taking into account the risk of the injury or condition represented by the CPT code using a table of CPT codes and their assigned risks, and a separate table with the risk levels sufficient for the expected competency level of the practitioner to allow the practitioner to perform competently. Within the dotted lines 600 are the assigned services 537 for the prototypical PBSCP as described above. The services on the outside of the dotted line are applied to extraordinary care provided by the extraordinary specialist 340. The resulting service definition then is used by the providers to acquire or maintain the necessary equipment, certifications, and training to perform them. For example, primary care providers as arranged for by the prior art do not typically possess an X-ray machine nor the training to use it. They rely on referrals to radiology. But the system disclosed can assign low-risk X-rays to PBSCPs, which would then require the PBSCP to acquire a simple X-ray machine (portable ones are affordable and easy to use) to do these low risk X-rays in house, as well as to be trained on the system and have the portable X-ray integrated into the EHR. This way, a patient presenting with a minor broken wrist or arm can be diagnosed by the PBSCP in the office, and if the fracture is beyond the PBSCP's training or capabilities an immediate referral can be generated—with the radiology images that were now already captured—to the CSP or ESP. Furthermore, the embodiment used to generate 600 used the procedure codes encompassed by 600 to generate the expected market value of the services provided, by referring to the Medicare reimbursement rates for each of the included services, and thus established using an actuarial model based off of typical working age populations the likely market value of the services provided, and thus produced a recommendation for the annual subscription fee necessary to enable the provider to provide the identified procedures. Some embodiments use the price the patient group will bear to determine the services: one uses a risk-price combined metric, such as ordering the procedures ranked first by risk then by price and choosing from the list bottoms up the procedures which, when entered into the actuarial model, produce a price just at the limit of what the group will bear.

Adequate funding is a reason why the prior art has failed to address the problems described herein. To address that and ensure that there are multiple adequate funding operations to ensure a baseline of available and effective treatment by the practitioners, multiple embodiments are disclosed that have alternative ways or variations on funding, a key piece to the process of determining services offered or available.

Some embodiments operate not on a fixed fee for the entire service but by having a balance of primary and basic specialty care subscription along with an insurance catastrophic or stop-loss style policy to cover catastrophic care. This produces a wrapped insurance/subscription product. (Note: by “wrapped” here, we mean an insurance policy that covers the residual risk not covered by another product, and not the investment insurance wrapper, an entirely different concept.) This wrapped (or combined, or consolidated) product has the advantages of up-front funding of the fixed and likely variable cost of the vast majority of care, with differentiated actuaries for emergency and acute care. For payors, the expected overall cost drops substantially, as the capitated funds and provided services absorb the variable costs for chronic care. For the provider, emergency and catastrophic care—which is highly variable—may not need to be absorbed, as those services do not lend themselves to capitation. For the patient, they may have front-to-back treatment through one consolidated medical care product. Note that the actuaries for the catastrophic and emergency care have the form of hit-or-miss statistics: the combination of the two distributions, one of approximately 0 and the other, with some residual probability dependent on the patient's age and preexisting conditions, a high-mean, high-variance catastrophic payout. (The mean tends to rise as the patient ages or has more diagnosed conditions; the variance may rise or fall.) These sorts of remainder or residual risks are well suited to reinsurance: because of the residual probability, a reinsurer can charge a number less than the mean of the overall distribution but greater than the product of the mean times the residual probability (or the expected cost of the residual risk), and take all the risk, or can take a second residual by only insuring the catastrophic risk above some number and leaving the initial catastrophic payout in the hands of the payor, thus capping upside risk with low administrative costs. In other words, this risk appears as a typical reinsurable risk: the reinsurer takes a payment and does nothing in almost all cases, as the likelihood of being called on the second residual risk is very low, an overall lower-mean, high-variance outcome and a left-leaning distribution. This is quite different from standard capitation practitioners seeking reinsurance, because their risk statistics are primarily dominated by exceeding a low capitation payout when the population's chronic, not catastrophic, illness rates increase above expectation, a highly likely event with a stable (and thus aging) population.

Another embodiment is for the provider to be the plan administrator, take the entirely of the payments, and offer the combined product by outsourcing the wrapper insurance policy to reinsurance. Another embodiment is for the policy provider to marry the capitation product with existing catastrophic and hospitalization policies, rather than seeking new ones: AFLAC and others offer these policies directly to companies or organizations to provide to their members. Marrying the catastrophic and hospitalization policies provides a cost-efficient way of covering the second residual risk—and some of the first residual risk—without seeking a reinsurance policy that may or may not be written. It's important to distinguish the difference between the provider and the self-insured payor being the policy provider, as the self-insured payor may already have these policies in place but has been traditionally not seeing them in use or only offering them optionally, but now can offer them mandatorily (and thus shifting some premiums away from catastrophic procedure reimbursement to catastrophic insurance premiums) with a nearly free hand in how they want to handle that risk balance. Obviously, an insurance carrier serving as the policy provider will not need to marry off-the-shelf catastrophic insurance, as they would likely rather absorb that into their actuaries and instead have a general reinsurance policy against their extreme risks if they wish those to be covered at all, at their scale.

For an employer, one embodiment is constructed as follows: the employer offers a health insurance package. It looks vaguely like a traditional HMO, but it has unlimited doctor's visits to the medical provider with no copays, no fees, free basic labs, free generic pharmacy, and unlimited referrals (but no self-referrals) to specialists in a broadest network of one or more standard PPO insurance carriers. This is not a PPO. There is no pharmacy coverage outside of the provider. Most services will be handled by the provider. But the patient can go to any emergency room, and any hospital for emergency or acute treatment, and the provider will take care of the billing in its entirety. The patient sees either nothing, or if the employer or plan provider wishes, a small coinsurance from the entire extraordinary service. One embodiment of this method is that the external plan provider is named as the administrator representing the responsible party (the patient for copays and coinsurance, the plan provider for the bulk), and handles distributing the billing and reimbursement of fees from the ultimate payors. Another embodiment is for the plan provider and employer or group purchaser to form a joint venture to stand in the shoes of the provider and employer.

For an individual plan, one embodiment is as follows: the patient purchases from the plan provider or its administrator a higher subscription fee, for an all-inclusive package with urgent, catastrophic, and primary and specialty care. This package may or may not have copays and coinsurance for the residual risks. The provider or its administrator is listed as the billing contact—on the patient's behalf—for the non-provider-offered services, and the patient is given a special insurance card that shows as having only the ultimate copay or coinsurance. The provider then is listed as a beneficiary on behalf of the patient for the wrapper catastrophic and hospitalization insurance policies, that it offers to its members through a membership model (such as a PEO or mutual benefit organization, based in part on the needs of the catastrophic insurer). As the patient is referred out to additional service outside of the medical provider, the plan provider will file the claim against the catastrophic insurance policy if applicable, accept the reimbursement and use that to defray the costs owed under the “insurance card”. With that method, the plan provider is floating the payment risk. Another embodiment is for the plan provider to accept and be granted limited representation of the patient, so that the patient remains on the hook for claim payment failures but has the paperwork handled by the plan provider as an administrative convenience. This this embodiment, the patient may request a complete breakdown of the combined policy into its constituents, as the patient has a legal right to know what is covered and what is not. The plan provider offers as a service access to a user-friendly customer service department, who has the job of explaining exactly how this works, over the phone and electronically, and for presenting what the likely risk exposure of the patient is for any given procedure. One embodiment of the latter uses the outcome risk of the analysis engine above to help produce a distribution or range of possible payouts. In any case, it is considered a service the plan provider offers to make the overall package less obscure and more understandable. (Note that the same service may be offered to employers for their group plans, above, as a value-added service of the plan provider.)

Obviously, subsets of the above, or added fees, can be introduced as further embodiments with no further innovation.

Another embodiment has the individual payor retain all of the risk by being the direct purchaser and act as its own agent, but with a personal representative (live or bot) of the plan provider either in person, online, or over the phone walking the payor through the necessary steps to do this themselves. One embodiment is for the representative to coordinate with the payor through a joint user interface, where the online forms and computer systems the payor must navigate through the external providers is presented alongside with instructions derived automatically or by the representative to show them what to do next and what to enter. A further embodiment is for the representative to take partial or complete control over the payor's own access to the external systems and to circle or otherwise highlight or distinguish what the next steps are, in an assistive fashion. Another embodiment is for that assistance to include auto filling forms, auto scrolling, and highlighting the parts where the payor must present her signature or affirmation.

In all cases above, the plan provider can be the same as the medical provider or a separate or affiliated entity.

As a part of defraying costs or for convenience, employer sponsors have the option of providing real estate for the medical practice, and additionally may choose to limit some or all services to the on-site clinic.

For self-insured or small group insured organizations, in one embodiment the plan provider serves as an aggregator, aggregating and mutualizing the risk pools. In some sense similar to a PEO, which aggregates HR interests among small companies to produce a larger pool, the aggregation here allows the smaller payors to have access to more distributed risks and thus lower variable costs. In one embodiment, the aggregator forms a multiple joint venture between the aggregated entities, with established capital call responsibilities and risk limitations to mitigate aggregated losses. In another embodiment, the aggregator offers group reinsurance or catastrophe insurance, possibly resold, to the joint venture or individual entities, or both. The plan provider may still offer off-the-shelf catastrophic wrapper consolidation.

Another embodiment is for the plan provider to use a computer to calculate the actuarial risks based on the patient populations from the aggregated EMR, mooted population statistics, and payor-provided information, using the above methods to calculate the overall risk, the residual and retained risks, and to offer a list of possible plans and products to manage that risk. One further embodiment is to use genetic risks (one can see these easily enough today on Promethease after taking a genetic test from providers such as 23andme) to inform the patient risk statistics using statistical analysis. One further embodiment is to use epigenetics and cross-population rates of incidence, using aggregated EMR data to perform statistical correlations (such as a logistic regression, or to use neural network learning) and assign probabilities or risks. To perform the risk calculations, one method is for the computer to use the statistical distributions expected for each possible offered product, along with the external and internal prices for each point in the distributions using internal and partner pricing models and showing side by side the different options. For example, if the patient population is 500 (50%) people over 50 years old, and the external catastrophic insurance provider has a pricing model that has been deduced or learned from the provider to charge $5000/yr for people over 50 and $3000/yr for people under 50 for a 30-day hospitalization policy, with the likelihood of the 50+ cohort to exceed 30 days of coverage to be 10% per person with a $50 k expected charge per excess, with 0% for the under 50 cohort, and the internal capitation prices to be offered are $3000/yr per patient regardless of age, the computer will offer the choices of $3000*1000 people=$3 m for pure primary and basic specialty care, or $5000*500+$3000*500+$3 m=$7 m/yr for the 30 day policy with excess risk of 10%*500*$50 k=$2.5 m.

In one embodiment, a franchiser franchises the medical provider methods and systems to groups that wish to enter the medical provider business under this model. In one embodiment, the franchiser is or affiliates with the plan provider to handle the billing and administration. In another embodiment, the franchiser retains or refranchises out the CSPs and central functions (such as mail-order pharmacy, centralized and exotic lab processing) and offers access to that as part of the franchise service. In another embodiment, the franchiser offers the EMR and related electronic and telepresence services to the franchisee. In another embodiment, the franchiser offers uniform patient access to the EMR as a service. In another embodiment, the franchiser offers booking and optional medical networking services to the franchisee, freeing them to focus entirely on the practice. (This is basically a practice in a box.)

In one embodiment, the franchisee is a group practice, and is responsible (with or without the franchiser's real estate help) to procuring suitable real estate, medical equipment (again whether distributed by the franchiser or through local distributors), and utilities. In another embodiment, the physician interfaces with the franchiser as the franchisee, and the franchiser provides hoteling space in a suitable practice site and access to equipment.

In all cases, the fees may be a fixed fee which may either be collected in advance or arrears or may be deducted from a revenue-share component of the franchise, where the franchiser is responsible for collecting the overall revenue and disbursing it as profits and salary to the franchisees. With these embodiments, both employee and independent contractor franchisees are possible: for employees, the distributions have the character of revenue share; for independent contractors, the distributions are sent with 1099 s. In all cases, the franchiser maintains through its standards required upon the franchisees and the services it provides a seamless user experience for the patient and payor.

For enterprise payors, the enterprise may wish to engage with a franchisor as a franchisee for the entire services, in which case they are able to retain the fees paid in as per-service profit and instead agree on an advance profit share. This is appropriate in many cases, and likely in cases where the employer provides the real estate. Another embodiment is for the enterprise to serve as a subfranchiser, and offer the space as a clinic that either they or the main franchiser may staff with franchisee providers and physicians.

One embodiment gives physician members of the franchise reciprocal practicing privileges at other franchisees, either restricted to networks or globally. Another embodiment is to grant physician franchisees fractional membership with multiple franchisers or subfranchisers, and thus have a patient load secured through multiple locations with the option to hotel or offsite for some patients here and others there.

Another embodiment is that an existing pharmacy or retail providers engages as a franchisee or subfranchiser. As a group practice franchisee, they gain the benefits of providing medical service and having a stable network of customers, with the profits of the service accruing to them and with their physician employees seeing a benefit of being a member of a larger physician network. The various methods and embodiments are designed so that many are flexible. In all cases, however, the franchiser may retain “corporate owned” practices and serve as its own franchisee, or even subfranchisee with interjected external subfranchisers.

For many of the reimbursement embodiments, it is important to note that the methods and unique insurance or funding products takes into account the unique distribution of services across the different service providers. The hit-and-miss statistical nature that allows catastrophic/stop-loss/reinsurance polices to operate requires that the care be provisioned so that substantially more than traditional primary care is provided by the first layer of medical services (PBSCP); otherwise, there is no good way to distinguish between a referral to a high cost specialist for a routine PBSCP-providable treatment (say, ringworm evaluation) and a high-risk evaluation (third-degree burns), both of which would usually occur in the same procedure code for the office visit. (Diagnostic codes are not sufficient, as EHRs often do not reliably tie the diagnostic code to the instance of the procedure in a manner that would sufficiently allow the rearrangement of services to ensure the hit-or-miss nature be largely true: too many would slip through.) The central system for service assignment allows both medical employees and franchisees to provide treatment to the level of their abilities consistent with the needs of the patients in the plan and the policies of the plan; without that system, the employee or franchisee could not become a PBSCP, CSP, or ESP, but would instead default to the posture of the prior art and not be educated enough or have the appropriate equipment in most cases to perform the services requested.

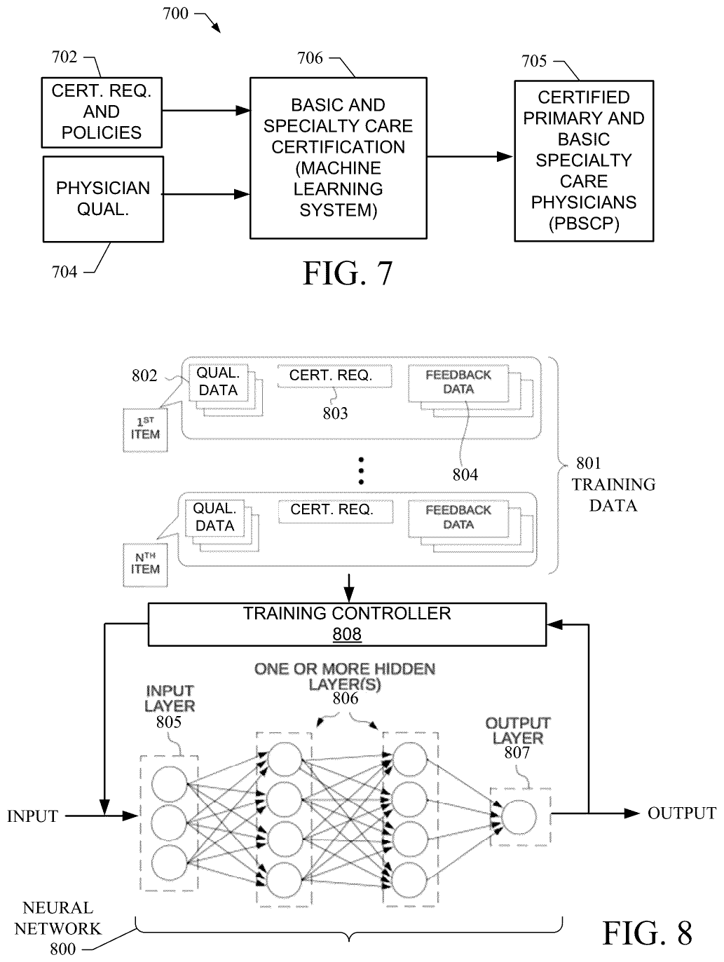

FIG. 7 shows a diagram of a certification system 700 that utilizes a machine learning system to certify primary and basic specialty care physicians. In one embodiment, certification requirements and policies 702 are input to a basic and specialty care certification machine learning system 706. Physician qualifications 704 for all physicians applying to be certified are also input to the basic and specialty care certification machine learning system 706. The machine learning 706 operates to determine a certification level for each physician based on the certification requirements 702 and physician qualifications 704. The system is able analyze large numbers of certification requirements and physician qualifications when making certification determinations. Thus, hundreds or thousands of physicians can be certified and re-certified as their qualifications and the certification requirements change.

In one embodiment, the certification results for each applying physician is determined as either “certified” or “not certified.” In another embodiment, multiple levels of certification are possible, and each physician is certified at a selected level based on the physician's qualifications. In one embodiment, a neural network is utilized for the machine learning system but in other embodiments, any suitable machine learning system can be utilized.

FIG. 8 shows a diagram of the neural network 800 for use as the machine learning system 706 shown in FIG. 7. In one embodiment, the neural network 800 is trained using training data 801 that includes multiple instances of training records that include physician qualification data 802, certification requirements 803, and feedback data 804. A training controller 808 operates to utilize the training data 801 to train the neural network 800, which comprises an input layer 805, one or more hidden layers 806, and an output layer 807. Once trained, the neural network 800 accepts inputs, such as the certification requirements 702 and the physician qualifications 704 to generate the certified primary and basic specialty care physicians 705 at an output.

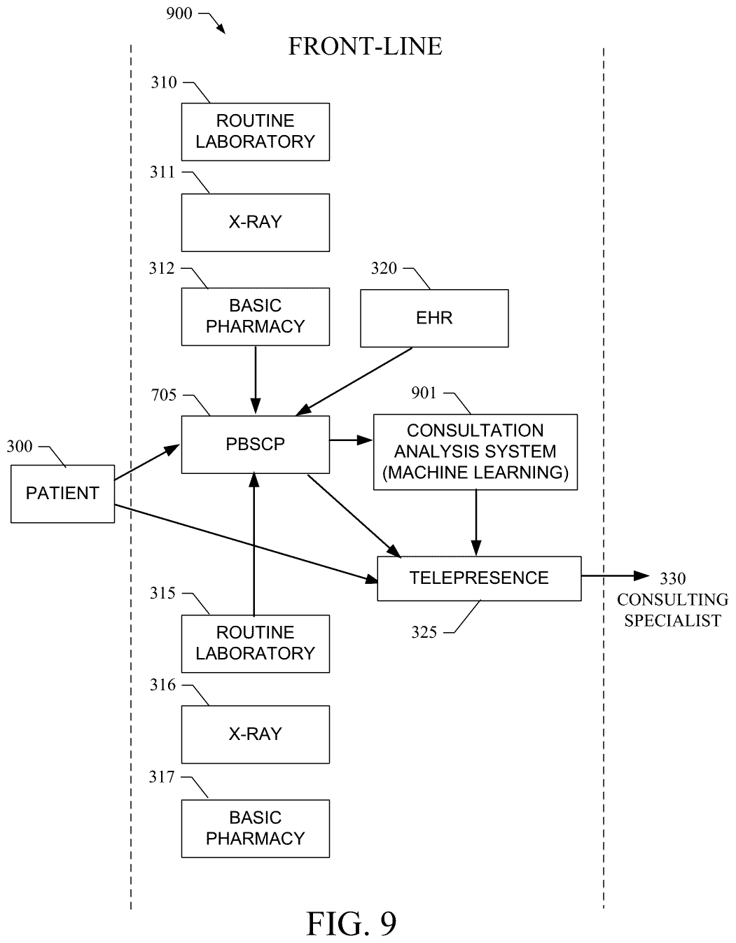

FIG. 9 shows an alternative embodiment of a front-line processing system 900 for use in the embodiment of the system shown in FIG. 3. The front-line processing system 900 includes many of the functions as shown in FIG. 3, but also includes certified PBSCP 705 as determined from the system shown in FIG. 7. The certified PBSCP interact with the patient to provide primary care in addition to basic specialty care for which the physician is certified. The results of the physician's evaluation, diagnosis and treatment are forward to a consultation analysis system 901. The physician's certification level is also provided to the system 901. The system 901 that utilizes machine learning to determine when a consultation with a specialist is required based on the information it receives. When it is determined that a specialist consultation is required, the system 901 initiates the consultation using the telepresence system 325. For example, if the patient's symptoms indicate that a serious condition may exist that is beyond the expertise of the certified PBSCP, the system 901 automatically initiates a consultation using the telepresence system 325. In one embodiment, a neural network is utilized for the machine learning system 901 but in other embodiments, any suitable machine learning system can be utilized

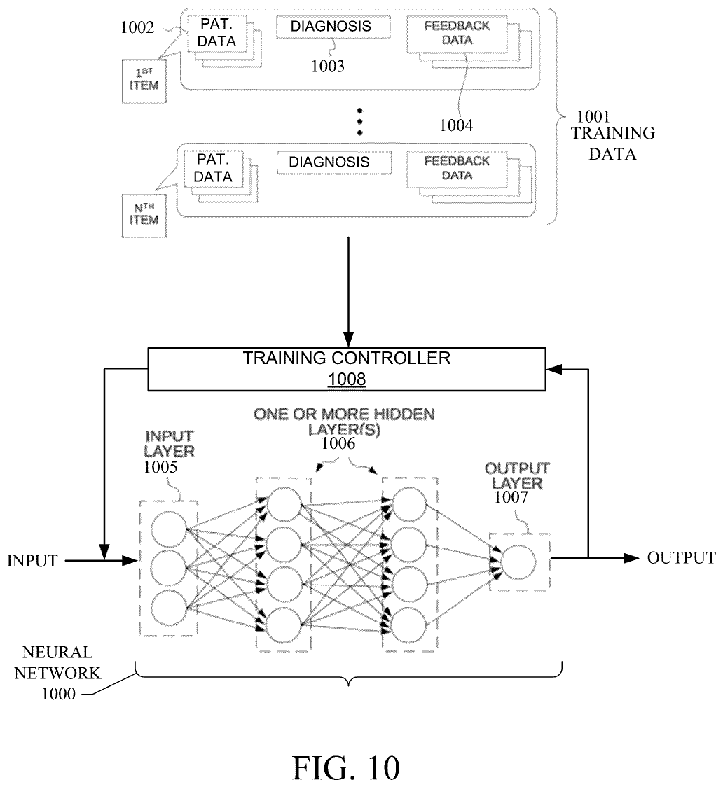

FIG. 10 shows a diagram of the neural network 1000 for use as the machine learning system 901 shown in FIG. 9. In one embodiment, the neural network 1000 is trained using training data 1001 that includes multiple instances of training records that include patient data 1002, physician diagnosis data 1003, and feedback data 1004. A training controller 1008 operates to utilize the training data 1001 to train the neural network 1000, which comprises an input layer 1005, one or more hidden layers 1006, and an output layer 1007. Once trained, the neural network 1000 accepts inputs, such as the patient data 1002 and diagnosis data 1003 to determine when to initiate a specialist consultation. For example, the output of the network 1000 is input to the telepresence system 325 to initiate the specialist consultation.

In the description herein, one or more embodiments of the invention are described, with process steps and functional interactions. Those skilled in the art would realize, after perusal of this application, that embodiments of the invention might be implemented using a variety of other techniques not specifically described, without undue experimentation or further invention, and that such other techniques would be within the scope and spirit of the invention. The use of the words “can” or “may” in regards to the structure and operation of embodiments is to be construed as referring to further embodiments and configuration options, and does not require further experimentation or invention.

The scope and spirit of the invention is not limited to specific examples disclosed therein but is intended to include the most general concepts embodied by these and other terms.

Although the invention has been described with reference to several exemplary embodiments, it is understood that such descriptions and illustrations are not limiting. Changes may be made within the purview of the appended claims, as presently stated, without departing from the scope and spirit of the invention in its aspects. Although the invention has been described with reference to particular means, materials, machines, and embodiments, the invention is not intended to be limited to the particulars disclosed; rather, the invention extends to all functionally equivalent structures, methods, machines, and uses such as are within the scope of the invention and claims.

What is claimed is:

1. A method of making specialty medical treatment available to patients, comprising steps of:
operating a first machine learning system to certify primary and basic specialty care providers;
assigning to the patient a primary and basic specialty care provider that provides primary care and basic specialty care procedures and services,
operating a computer to calculate fees for the primary care and basic specialty care procedures and services based on patient populations,
providing to the patient, by the assigned primary and basic specialty care provider, any of the primary care and basic specialty care procedures and services,
operating a second machine learning system that automatically triggers a consultation with a consulting specialist provider based on the primary care and basic specialty care procedures and services provided to the patient; and
operating a telepresence system to conduct the consultation that provides communication between the patient, the primary and basic specialty care provider, and the consulting specialist provider to provide specialty medical treatment to the patient when the specialty medical treatment is not part of the primary care and basic specialty care procedures and services.

2. The method of claim 1 further comprising step of: referring the patient to an extraordinary specialist provider for services that the assigned primary and basic specialty care provider and said consulting specialist provider are at least one of: not capable of providing a treatment to the patient at the time, and needing a medical consultation from the extraordinary specialist provider.

3. The method of claim 1 wherein said fees for the set of assigned procedures and services are calculated at least in part from at least one of: service cost, service requirements, policies, the providers, the practitioners the providers have, said practitioners' skill levels and capabilities, services the provider is capable of offering at the moment, and services the provider can become capable of providing.

4. The method of claim 3 wherein the primary and basic specialty provider is required to offer a baseline of basic specialty procedures.

5. The method of claim 4 wherein said baseline comprises at least one of: injections, allergy testing, spirometry, skin excision and biopsy, laser treatment and removal, hypertension and cholesterol management, noninvasive cardiovascular testing, AICD and pacemaker management, casting and repair of minor bone fractures, minor orthotics, menopausal hormone replacement, diabetes management, dialysis, optometry, and providing corrective lenses.

6. The method of claim 1 wherein said patient is responsible for fees for enrollment include an amount related to the patient's portion of the fixed costs necessary to operate the assigned primary and basic specialty care provider.

7. The method of claim 6 wherein said fees for enrollment include an amount related to contributions to a risk pool sufficient to cover at least one of: the variable costs of the patient, and the costs for service of additional specialty providers.

8. The method of claim 6 wherein said fees for enrollment include an amount related to the premiums necessary to enable the procurement of insurance, comprising at least one of commercial insurance and enterprise self-insurance, to cover at least one of: the variable costs of the patient, and the costs for service of additional specialty providers.

9. The method of claim 8 wherein the patient portion of claims of said insurance is handled and at least in part paid by the administrator of said medical plan wherein the patient does not need to receive the demands for the patient portion of the claim when the administrator pays the patient portion of the claim in full.

10. The method of claim 1 wherein said consulting specialty provider performs consultations remotely with the patient and said primary and basic specialty provider using access to the patient's electronic health record.

11. A system of making specialty medical treatment available to patients comprising:
a first machine learning system that certifies primary and basic specialty care providers;
a primary and basic specialty care assigner, which assigns to a patient a primary and basic specialty care provider,
a services assigner, which assigns to the assigned primary and basic specialty care provider a set of procedures and services that comprise both primary care procedures and specialty care procedures that the provider can perform on the patient under a medical plan,
a computer to calculate fees for the set of procedures and services based on patient populations,
a second machine learning system that automatically triggers a consultation with a consulting specialist provider; and
a telepresence system to conduct the consultation that provides communication between the patient, the primary and basic specialty care provider, and a consulting specialist provider to provide specialty medical treatment to the patient when the specialty medical treatment is not part of the primary care and basic specialty care procedures and services.

12. The system of claim 11, wherein the referral system refers the patient to an extraordinary specialist provider for services that the assigned primary and basic specialty care provider and said consulting specialist provider are at least one of: not capable of providing a treatment to the patient at the time, and needing a medical consultation from the extraordinary specialist provider.

13. The system of claim 11, wherein services assigner calculates said set of assigned procedures and services at least in part from at least one of: service cost, service requirements, policies, the providers, the practitioners the providers have, said practitioners' skill levels and capabilities, services the provider is capable of offering at the moment, and services the provider can become capable of providing.

14. The system of claim 13, wherein the primary and basic specialty provider is required to offer a baseline of basic specialty procedures.

15. The system of claim 14, wherein said baseline comprises at least one of: injections, allergy testing, spirometry, skin excision and biopsy, laser treatment and removal, hypertension and cholesterol management, noninvasive cardiovascular testing, AICD and pacemaker management, casting and repair of minor bone fractures, minor orthotics, menopausal hormone replacement, diabetes management, dialysis, optometry, and providing corrective lenses.

16. The system of claim 11, wherein said fees include an amount related to the patient's portion of the fixed costs necessary to operate the assigned primary and basic specialty care provider.

17. The system of claim 16, wherein said fees include an amount related to contributions to a risk pool sufficient to cover at least one of: the variable costs of the patient, and the costs for service of additional specialty providers.

18. The system of claim 16, wherein said fees include an amount related to the premiums necessary to enable the procurement of insurance, comprising at least one of commercial insurance and enterprise self-insurance, to cover at least one of: the variable costs of the patient, and the costs for service of additional specialty providers.

19. The system of claim 18, wherein the patient portion of claims of said insurance is handled and at least in part paid by the administrator of said medical plan wherein the patient does not need to receive the demands for the patient portion of the claim when the administrator pays the patient portion of the claim in full.

20. The system of claim 16, wherein said consulting specialty provider performs consultations remotely with the patient and said primary and basic specialty provider using access to the patient's electronic health record.
